(19) United States
(12) Patent Application Publication
(10) Pub. No.: US 2011/0034926 A1
(43) Pub. Date: Feb. 10, 2011

(54) BIORESORBABLE MATERIAL

(76) Inventors: Christin Menneking, Hannover (DE); Dirk Borman, Sehnde (DE); Peter Behrens, Mellendorf (DE); Friedrich-Wilhelm Bach, Isernhagen (DE)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 12/988,055

(22) PCT Filed: Apr. 17, 2009
(86) PCT No.: PCT/EP2009/002829

§ 371 Date: Oct. 15, 2010

(30) Foreign Application Priority Data
Apr. 18, 2008 (DE) 10 2008 019 748.3

(51) Int. Cl. A61B 17/68; A61K 9/00; A61K 33/00; A61P 19/08; A61B 17/72

(52) U.S. Cl. 606/62; 424/400; 424/722; 606/329

(57) ABSTRACT

The present invention in a first aspect relates to an element having a nonporous hollow body and a filling, wherein the nonporous hollow body is formed from bioresorbable magnesium and/or a bioresorbable magnesium alloy and the filling comprises a biocomposite material, wherein the biocomposite material comprises at least one biocompatible polymer component A and one ceramic component B. In a further aspect, the present invention is directed to a method for producing this element, which is especially suitable for use in bone surgery.

The present invention relates in a first aspect to an element having a nonporous hollow body and a filling, wherein the nonporous hollow body is formed from bioresorbable magnesium and/or bioresorbable magnesium alloy and the filling comprises a biocomposite material, wherein this biocomposite material comprises at least one biocompatible polymer component and one ceramic component. In a further aspect, the present invention is directed to a method for producing this element, which is especially suitable for use in bone surgery.

### BACKGROUND OF THE INVENTION

Elements for use as implants, in the field of bone surgery for example, have a broad range of use. Elements made of bioresorbable materials, elements made of nonresorbable materials, or combinations thereof are used depending on the application area.

Fully or partially bioresorbable implants in particular are increasingly used in bone surgery. These bioresorbable materials are corroded or otherwise degraded in the human body and disappear virtually without leaving a trace.

Medical implants of the type mentioned are known in various forms. They can be fastening elements for a bone, for example, plates, screws, or pins, surgical suture material, surgical meshes or films, or else prostheses.

The idea of using magnesium and magnesium alloys as bioresorbable materials has already been known for a long time. For example, surgical suture material made of magnesium and magnesium alloys was described in documents DE 676059, DE 665836, and DE 688616. However, the suture materials described therein, made of magnesium and magnesium alloys, have huge disadvantages in terms of gas evolution and uneven onset of corrosion.

EP 1 395 297 also describes medical implants for the human or animal body which are made of magnesium alloys that degrade in the body. Absorbable material comprising magnesium in particular is considered suitable for such implants. However, these implants based on magnesium have disadvantages when used in the body. These disadvantages include a relatively large amount of gas being produced per unit time, more particularly hydrogen. This results in gas pockets in the body, and the materials themselves are degraded unevenly.

Materials used as an implant firstly have to be inert to the extent that no rejection or inflammation reactions are induced in the living organisms and the materials induce a negative effect on the recovery of the tissue or the entire organism. Secondly, the materials of the implant have to satisfy a very wide range of different mechanical requirements, such as a high load-bearing capacity. Especially when used in bone, as a bone pin or intramedullary pin for example, the implant firstly has to have a high rigidity in order to sufficiently strengthen the bone. Secondly, however, the implant has to demonstrate a sufficient flexibility, i.e., it must not be too brittle in order to prevent a load causing snapping.

There has to be a sufficient bendability, ductility so that the material does not lose its shape with a steady load over a period of time.

Currently, elements useful as resorbable implants are produced from different resorbable materials. For instance, bioresorbable polymers are used in bioresorbable implants. These polymers have a good biocompatibility, but have only a low mechanical load-bearing capacity and hardness, and they are therefore useful as bone substitute material only to a limited extent. They cannot be implanted at sites subjected to strong mechanical loads.

Alternatively, ceramic implants have been proposed. Ceramics generally have a high hardness. However, they are mostly brittle and easily snap. Especially the ease of snapping leads to problems, since fragments may move uncontrollably in the tissue and may thus lead to complications. In addition, ceramic implants dissipate rather more slowly. It may take years before a natural bone has reformed.

Mixing the two materials mentioned, bioresorbable polymers and ceramic implants, results in composite materials which are likewise used as implant materials. For instance, EP 289 562 describes such biocomposite materials which are for use in bone surgery and which consist of a bioceramic portion and a polymer portion which is bioresorbable. However, such materials likewise do not satisfy the mechanical requirements of implants, more particularly in the field as bone substitute materials, such as a bone pin, more particularly an intramedullary pin.

For these applications, metallic implants made of bioresorbable materials have been proposed as further possibilities. More particularly, the bioresorbable metallic implants made of magnesium should be mentioned here. A further advantage of these magnesium-containing implants is the stimulation of bone growth by magnesium. Such metallic implants are described in, for example, EP 1 338 293 or EP 1 395 297. The major disadvantage of these implants is the hydrogen generated within the body during the corrosion; in order to resolve this problem, a reduction in the amount of magnesium was considered. For example, it was proposed to use magnesium sponges. Owing to the high surface area, this results, however, in an accelerated corrosion, and so durability is not ensured. A further disadvantage of these sponges or other open-cell or porous structures is the likewise increased release of hydrogen. Accordingly, implants as described in the subsequently published application WO 2008/064672 A2 and having an open-cell metal structure, for example, are likewise not useful.

An object of the invention is to provide an element, more particularly a medical implant, instrument, or auxiliary usable in the human or animal body, which avoids the abovementioned disadvantages and is degradable with only minimal secondary effects, if any, in the body, with this element, more particularly this implant, withstanding the mechanical loads it is subjected to during use.

### DESCRIPTION OF THE INVENTION

This object is achieved by the element according to the invention having a nonporous, more particularly non-open-cell, hollow body and a filling. The hollow body is formed from bioresorbable magnesium and/or a bioresorbable magnesium alloy, whereas the filling comprises a biocomposite material which comprises at least one biocompatible polymer component and one ceramic component. Furthermore, there is provided a method with which the elements according to the invention are obtainable.

In a first aspect, the present invention is directed to an element having a hollow body and a filling, wherein the hollow body is formed from bioresorbable magnesium and/or a bioresorbable magnesium alloy and the filling comprising a biocomposite material, wherein the biocomposite material comprises at least one biocompatible polymer component and at least one ceramic component.

The element according to the invention, also referred to as a structural element, is preferably one which consists of the nonporous hollow body and the biocomposite material. Preferably, the element according to the invention is an implant, instrument, or auxiliary, more particularly an implant, such as a bone pin or intramedullary pin.

This element, which is especially useful as an implant in particular as bone substitute material, is bioresorbable and combines the good mechanical properties of the individual constituents and also their bioresorbability in order to be used as, for example, bone substitute materials, such as a bone pin and intramedullary pin. The hollow body formed from magnesium and/or magnesium alloy is a biocompatible, coatable, degradable magnesium and/or magnesium alloy. In a preferred embodiment, the hollow body is formed from a magnesium alloy. The hollow body is a nonporous and, more particularly, non-open-cell hollow body made of magnesium or a magnesium alloy. As a result of the magnesium or the magnesium alloy being nonporous, the release of hydrogen is reduced owing to the small surface area. At the same time, the thickness of the hollow body can be reduced without affecting the stability too much. The hollow body can be a hollow body open on at least one side. Preferably, the hollow body comprising the biocomposite material is closed.

Preferably, the nonporous hollow body is made of a magnesium alloy having a proportion of magnesium greater than 50% by weight, such as 60% by weight, 70% by weight, 80% by weight, and more preferably 90% by weight. Useful alloys are, for example, LANd442 (lithium, aluminum, neodymium; 4%, 4%, 2% by weight) or ZM21 (zinc, manganese; 2%, 1% by weight).

This coatable magnesium and/or magnesium alloy surrounding the filling and forming the nonporous hollow body fulfills various purposes. Owing to the good mechanical properties, the element can simply be implanted in the form of an implant. Thus, these implants can also be used at sites which are exposed to strong mechanical loads. The presence of magnesium additionally promotes new bone formation at the implant. This leads to a rapid integration of the implant into the surrounding bone. The rapid growth of new bone at the implant is an important aspect for the healing and the rapid requirement of load-bearing capacity of the bone.

In a preferred embodiment, the hollow body is coated on its external side, which is facing the tissue.

This coating can include, more particularly, bone substitute and/or pharmacologically active substances. This is understood to include, for example, pluripotent cells, bone marrow cells, or bone grafts which promote the ingrowth of bone into the degrading element.

More preferably, the hollow body is at least partially coated with growth factors which promote the ingrowth of bone. Alternatively, the hollow body is at least partially coated with active ingredients which improve tissue regeneration.

In a further preferred embodiment, the hollow body is endowed with a rough surface to further promote the ingrowth.

The filling of the element according to the invention comprises a biocomposite material. This biocomposite material is composed of at least one biocompatible polymer component and at least one ceramic component.

This filling makes it possible to strengthen the mechanical load-bearing capacity of the hollow body, more particularly to improve the rigidity and strength but also the ductility of the hollow body. At the same time, however, the danger of too strong an evolution of hydrogen during corrosion of the magnesium or the magnesium alloy is reduced. The structure according to the invention of the element makes it possible to keep the amount of magnesium in the element low without substantially impairing the mechanical properties of the element. For the filling, a multiplicity of materials is possible. Generally, biocompatible, compact or porous composite materials, bone cement, and functionalized materials are usable as a filling. Preferably, the biocompatible polymer component is selected from the group consisting of polysaccharides, polyglycolide; polylactide, glycolide/lactide copolymer, glycolide/trimethylene carbonate copolymer, poly-β-hydroxybutyric acid, poly-β-hydroxypropionic acid, poly-β-hydroxyvaleric acid, PHBA/PHVA copolymers, poly-p-dioxanone, poly-1,4-dioxanone-2,5-dione, polyesteramide, poly-ε-caprolactone, poly-δ-valerolactone, polycarbonate, polyether esters of oxalic acid, glycol esters, dihydropyran polymers, polyether esters, cyanoacrylate, collagen and derivates thereof, cellulose derivatives, and chitin polymer.

The polymer component can be present in, for example, fiber form. Particularly preferably, the biocompatible polymer component is a polysaccharide selected from chitin and chitosan, in the form of fibers for example. The filling has a ceramic, biocompatible component as a further constituent. This biocompatible, ceramic component is preferably selected from the group consisting of apatite, hydroxyapatite, fluorapatite, calcium phosphate, tricalcium phosphate, dicalcium phosphate, magnesium calcium phosphate, mixtures of hydroxyapatite and tricalcium phosphate, aluminum oxide ceramic, bioglass, glass ceramic which comprises apatite, and calcium carbonate.

Especially the use of chitosan-hydroxyapatite composite materials as a filler is advantageous. This filler is biocompatible and endows the hollow body with the required mechanical load-bearing capacity. The strength of this filler is, for example, within the range of normal bones, and these materials are therefore outstandingly useful as bone implants and, more particularly, as an intramedullary pin.

In a further preferred development of the invention, the filler comprises bone-formation-promoting factors, more particularly growth factors. The filler may comprise other factors which support the regeneration of the bone or the tissue. The filling may optionally consist of porous material.

Such elements, formed from a magnesium alloy hollow body and a chitosan-hydroxyapatite composite material filling, elements in the form of bone substitute materials or a bone pin for example, are completely resorbable in the body of humans and animals. Furthermore, they combine the positive properties of metallic implants with those of resorbable composite materials.

A further aspect of the present invention is directed to a method for producing the elements according to the invention. This method comprises the steps of providing a hollow body which is open on at least one side, such as on 2 sides. This hollow body is formed from magnesium and/or magnesium alloys. Subsequently, this hollow body is filled with the above-described filling which comprises a biocomposite material. This filling in the hollow body is optionally further compacted by suitable means. Subsequently, an insert is introduced into the opening(s) of the hollow body. This insert can, for example, be used to compact the filling, as mentioned above, in the hollow body. The insert, which is likewise formed from magnesium and/or magnesium alloy, is shaped such that it protrudes outward beyond the hollow body after introduction into the opening of the hollow body.

The insert protruding beyond the hollow body is then, in the section in which the insert is introduced into the hollow body, joined to the hollow body. Joining is understood to mean more particularly the joining process referred to in DIN 8593, for example soldering, adhering, or welding. Preference is given to inductive welding or laser welding. Particular preference is given to a WIG welding method. During joining, the insert projects outward beyond the hollow body. This protrusion makes it possible for the heat generated during, for example, welding to be dissipated outward. Otherwise, the filling would be exposed to too strong a heat, destroying the filling or changing it such that it is no longer biocompatible. Thus, when using a chitosan-hydroxyapatite filling for example, it must not exceed a temperature of 80° C., otherwise it will be changed such that it is no longer biocompatible.

The protrusion according to the invention of the inserts makes the required dissipation of heat possible, and the excessive heating of the filling material is prevented. At the same time, the insert is connected to the hollow body by a firm bond during joining.

After joining, the regions of the insert which project beyond the hollow body can be optionally removed, for example, by simply sawing them off. Alternatively, the protruding regions can be reshaped as a pin or another desired shape. Depending on the use, the element obtained can then be further processed, for example, the element can be appropriately adapted for use as an intramedullary pin or bone pin.

In a preferred development of the method, the hollow body comprises two opposite openings, and the filling is compacted or compressed by application of pressure onto the two inserts.

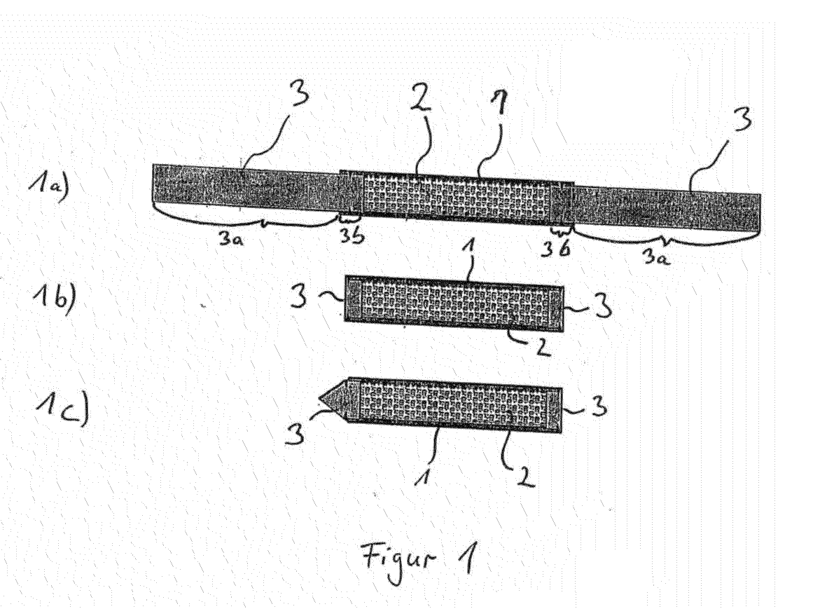

One embodiment of the invention is explained in detail below by reference to FIG. 1. FIG. 1 shows the different developments of the element at different time points of the method according to the present invention.

FIG. 1a shows the element according to the invention with a hollow body 1 and the filling 2. The hollow body has two openings into which the inserts 3 are introduced, sections 3b. The inserts 3 compact the filler 2 in the hollow body 1. The inserts 3 project beyond the filled body, section 3a. In this arrangement, the inserts 3 are then joined to the shaped body 1 in regions of the sections 3b, for example, by means of WIG welding methods. Subsequently, the sections 3a of the inserts 3, said sections protruding beyond the shaped body 1, can be completely or partially removed, for example, by sawing off the regions sticking out, in order to obtain an element as shown in FIG. 1b.

This element according to FIG. 1a or 1b can then be further processed, for example, to form a pin as shown in FIG. 1c.

By way of example, the production of a biocomposite material suitable for use as a filling for the element according to the invention is described below.

2 g of chitosan (low, middle, and high viscosity, Fluka) are dissolved in 2% acetic acid (Riedel-de Haën, puriss.). To this, 200 ml of 0.108 M Ca(Ac)₂ (Riedel-de Haën, puriss.) solution, 200 ml of 0.0648 M KH₂PO₄ (Merck, p.a.) solution, and 2 g of K₂CO₃ (Fluka, puriss.) are added in succession. The pH is adjusted to pH 9 with 1 M KOH (Fluka, ultra). The solution is stirred overnight and then centrifuged down and washed with water. The precipitated composite is added to a glass tube by mechanical compaction and dried at room temperature.

This filling consisting of a chitosan-hydroxyapatite mixture can then be cold extruded and introduced into the hollow body or shaped body 1. Subsequently, the shaped body containing the chitosan-hydroxyapatite filling is further treated, as described above under FIG. 1.

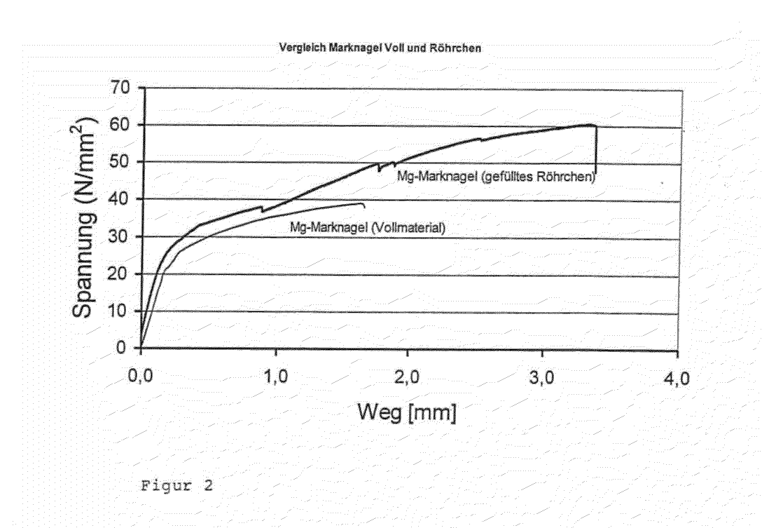

FIG. 2 shows the results of a determination of the bending stress of intramedullary pins. An intramedullary pin according to the invention comprising a ZM21 shell body having an above-described chitosan-hydroxyapatite filling is compared with an intramedullary pin comprising a LANd442 solid material. The differences are distinctly visible. The load-bearing capacity of the pin according to the invention is distinctly improved compared with the pin composed only of a magnesium alloy. The bending stress was tested with a 3-point bending test. Sample holder: 3-point bending beam; test velocity: 1 mm/min; support width: 15 mm; initial load: 2.5 N.

The bending strength of the pin according to the invention was likewise increased (273 N/mm² compared with 245 N/mm²).

### REFERENCE SYMBOLS LIST

1. Hollow body
2. Filling
3. Insert
3a. Section of the insert 3, said section protruding from the hollow body
3b. Region of the insert 3, said region being introduced in the hollow body

1. An element having a nonporous hollow body and a filling, wherein the nonporous hollow body is formed from bioresorbable magnesium and/or a bioresorbable magnesium alloy and the filling comprises a biocomposite material, wherein the biocomposite comprises at least one biocompatible polymer component and at least one ceramic component.

2. The element as claimed in claim 1, wherein the element is an implant, a medical instrument, or a medical auxiliary.

3. The element as claimed in claim 1, wherein the element is a medical implant used as a bone substitute material.

4. The element as claimed in claim 3, wherein the medical implant is a bone pin or intramedullary pin.

5. The element as claimed in claim 1, wherein the nonporous hollow body consists of a bioresorbable magnesium alloy.

6. The element as claimed in claim 1, wherein the biocompatible polymer component is selected from the group consisting of polysaccharides, polyglycolide; polylactide, glycolide/lactide copolymer, glycolide/trimethylene carbonate copolymer, poly-β-hydroxybutyric acid, poly-β-hydroxypropionic acid, poly-β-hydroxyvaleric acid, PHBA/PHVA copolymers, poly-p-dioxanone, poly-1,4-dioxanone-2,5-dione, polyesteramide, poly-ε-caprolactone, poly-δ-valerolactone, polycarbonate, polyether esters of oxalic acid, glycol esters, dihydropyran polymers, polyether esters, cyanoacrylate, collagen and derivates thereof, cellulose derivatives, and chitin polymer.

7. The element as claimed in claim 6, wherein the biocompatible polymer component is a polysaccharide selected from chitin and chitosan.

8. The element as claimed in claim 1, wherein the ceramic component B is selected from the group consisting of apatite, hydroxyapatite, fluorapatite, calcium phosphate, tricalcium phosphate, dicalcium phosphate, magnesium calcium phosphate, mixtures of hydroxyapatite and tricalcium phosphate, aluminum oxide ceramic, bioglass, glass ceramic which comprises apatite, and calcium carbonate.

9. The element as claimed in claim 1, wherein the filling of the hollow body and/or of the hollow bodies has bone-formation-promoting factors, more particularly growth factors, on its external side.

10. The element as claimed in claim 1, wherein the filling consists of porous material.

11. A method for producing an element as claimed in claim 1, comprising the steps of:
a) providing a nonporous hollow body open on at least one side,
b) filling the nonporous hollow body with the filling which comprises a biocomposite material,
c) compacting the filling in the hollow body,
d) introducing an insert into the opening of the hollow body,
e) joining the insert to the hollow body, characterized in that the insert protrudes outward beyond the hollow body during joining.

12. The method as claimed in claim 11, further comprising the step of
f) removing the region of the insert sticking out beyond the hollow body.

13. The method as claimed in claim 11, wherein the joining in step c) is a WIG welding method.

14. The method as claimed in claim 11, wherein the hollow body has two opposite openings and the filling is compressed by application of pressure on two inserts.
